(19) United States
(12) Patent Application Publication
(10) Pub. No.: US 2023/0404458 A1
(43) Pub. Date: Dec. 21, 2023

(54) GRAPHENE BASED ELECTRODE FOR ELECTROPHYSIOLOGICAL READINGS

(71) Applicant: University of Technology Sydney, New South Wales (AU)

(72) Inventors: Kimi Aki IZZO, New South Wales (AU); Mojtaba AMJADI POUR, New South Wales (AU); Shaikh Nayeem FAISAL, New South Wales (AU); Chin-Teng LIN, New South Wales (AU); Francesca IACOPI, New South Wales (AU)

(21) Appl. No.: 18/251,212

(22) PCT Filed: Oct. 29, 2021
(86) PCT No.: PCT/AU2021/051272

§ 371 Date: Apr. 28, 2023

(30) Foreign Application Priority Data
Oct. 30, 2020 (AU) 2020903960
Mar. 29, 2021 (AU) 2021900918

(51) Int. Cl. A61B 5/266; C01B 32/956; C01B 33/06; C01B 32/184; C01B 32/194; C30B 1/10; C30B 29/02; G01N 27/02

(52) U.S. Cl. CPC A61B 5/266; C01B 32/956; C01B 33/06; C01B 32/184; C01B 32/194; C30B 1/10; C30B 29/02; G01N 27/026; A61B 2562/125; C01B 2204/02; C01B 2204/22; C01P 2006/40; C01P 2004/03; C01P 2002/82; C01P 2002/85

(57) ABSTRACT

The present disclosure provides a graphene based dry electrode for electrophysiological readings, in particular for use with EEG, EKG, EMG, and EOG systems and a method for making said electrodes. The electrodes comprising a doped silicon substrate; a silicon carbide film on the substrate; a graphene surface on the silicon carbide film; wherein the graphene surface has undergone a functionalisation and/or intercalation process to increase the amount of oxygen functional groups present, said process being preferably carried out through repeated contact of the graphene surface with an electrolyte solution.

### TECHNICAL FIELD

The present disclosure relates to a graphene based dry electrode for electrophysiological readings, in particular for use with EEG, EKG, EMG, and EOG systems.

### BACKGROUND OF THE DISCLOSURE

A number of medical conditions can be diagnosed and monitored by obtaining and interpreting electrophysiological readings such as the electrical activity of various organs and locations. Examples include electroencephalography (EEG), electrocardiography (EKG), and electromyography (EMG) which measure the electrical activity of the brain, heart and skeletal muscles respectively. These techniques are non-invasive, which is to say that the electrical activity may be measured by placing electrodes in predetermined locations on the skin, without the need to make any incisions or otherwise harm the patient. For example, in EEG, a set of electrodes are placed on the scalp of the patient, sometimes in a net or cap, in EKG (also sometimes abbreviated as ECG), electrodes are placed on the limbs and torso of the patient, and in EMG, electrodes are placed around the muscle of interest. Similar equipment may also be used to perform electrooculography (EOG) which measures the corneo-retinal standing potential to track eye movements and/or responses to certain stimuli. This involves the placement of electrodes on the skin around the eye of a patient.

EEG also has applications in the creation of brain-computer interfaces (BCI), where an external device is controlled by the detection and translation of electrical signals produced by the brain of a user. In particular, BCIs enable the external device to receive and respond to the electrical signals produced by the action potential fired by groups of cells in the brain and the transduction of ion currents moving through brain tissue. EEG is particularly favoured for these applications due to its relative low cost and non-invasive nature, which is both safer for the user and easier than invasive reporting methods.

The electrodes used for non-invasive EEG applications can be divided into two broad categories: wet electrodes and dry electrodes. Wet electrodes, most commonly Ag/AgCl based electrodes, rely on an electrolytic gel being applied between the electrode and the skin of the patient. The electrolytic gel hydrates the uppermost layer of the skin and allows a conductive bridge between the electrode sensor and the ion currents in the brain tissue. Wet electrodes give low noise and low electrode-skin impedance, however they also have a number of disadvantages. The abrasive gel used is greasy and often uncomfortable for the user, the gel requires application and clean up between uses, and the conductive properties degrade as the gel dries out.

Dry electrodes, on the other hand, do not involve the use of a gel or other intermediate conductor between the skin and electrode, and may be placed directly on the skin. This makes dry electrodes easier to use, however present dry electrodes typically have a much higher impedance, and typically require more complex designs in order to account for this. This makes manufacture more difficult and potentially more costly as a result. Examples of dry electrode materials include conductive polymer foams made of urethane materials and spring-loaded pin electrodes, which use the spring to push against the skin and maintain contact.

Accordingly, there exists a need to provide for a dry electrode with a lower impedance and relatively easy manufacturability.

Graphene, a two dimensional form of graphite, has gained attention as a potential material for sensors for electrophysiological readings owing to its excellent mechanical strength and high electrical conductivity, as well as being chemically stable. Graphene has also been found to be biocompatible in a range of applications and can be cytotoxic against bacteria. Graphene is difficult, however to manufacture into the required geometries for integration into an electrode or other biosensor.

The present disclosure seeks to provide a graphene based dry electrode suitable for applications such as EEG which has equivalent or superior performance compared to existing dry electrodes.

### SUMMARY OF THE INVENTION

According to a first aspect, there is provided a process for forming a dry electrode for measuring electrophysiological readings, comprising: epitaxially growing a silicon carbide film on a doped silicon substrate; depositing at least two metals on a surface of the silicon carbide film, the at least two metals including at least one first metal and at least one second metal; heating the at least two metals, silicon carbide film, and substrate to cause the at least one first metal to react with silicon of the silicon carbide film to form carbon and at least one stable silicide, and the corresponding solubilities of the carbon in the at least one stable silicide and in the at least one second metal are sufficiently low that the carbon produced by the silicide reaction forms a graphene layer on the silicon carbide film; removing the at least one stable silicide and unreacted at least first and second metals to produce a structure having a doped silicon substrate and a silicon carbide film with a surface layer of graphene; repeatedly contacting the surface layer of graphene with an electrolyte solution to condition the graphene surface prior to use.

In some embodiments, the composition of the electrolyte solution is similar to human sweat.

In some embodiments, the electrolyte solution is phosphate buffered saline solution

In some embodiments, the concentration of the phosphate buffered saline solution is 0.01M.

In some embodiments, the electrolyte is human sweat.

In some embodiments, the corresponding solubility of carbon in the at least one second metal is lower than the corresponding solubility of carbon in the at least one stable silicide.

In some embodiments, the at least one first metal is nickel, and the at least one second metal is copper.

In some embodiments, the heating step is performed in an inert gas atmosphere.

In some embodiments, the heating step is performed under vacuum.

In some embodiments, wherein the vacuum has a pressure of about 10⁻³ to 10⁻⁵ mbar.

In some embodiments, the heating step is carried out above 800° C.

In some embodiments, the heating step is carried out at about 1100° C.

In some embodiments, the step of contacting the surface layer of graphene with the electrolyte solution is repeated more than three times.

In some embodiments, the step of contacting the surface layer of graphene is repeated between three and ten times.

According to a second aspect, there is provided a dry electrode for measuring electrophysiological readings, comprising: a doped silicon substrate; a silicon carbide film on the substrate; a graphene surface on the silicon carbide film; wherein the graphene surface has undergone a conditioning step of repeatedly contacting the surface layer of graphene with an electrolyte solution prior to use.

According to a third aspect, there is provided a dry electrode for measuring electrophysiological readings, comprising: a doped silicon substrate; a silicon carbide film on the substrate; a graphene surface on the silicon carbide film; wherein the graphene surface has undergone a functionalisation and/or intercalation process to increase the amount of oxygen functional groups present.

In some embodiments, the oxygen functional groups include C—OH and COOH.

In some embodiments, the functionalisation and/or intercalation process occurs substantially at the grain boundaries of the graphene surface.

In some embodiments, the substrate is attached to a metal pin button in a manner that enables electrical communication between the graphene surface and the metal pin button through the substrate.

In some embodiments, the substrate is attached to the metal pin button by a carbon tape.

In some embodiments, the electrode is produced by a process according to the first aspect.

According to a fourth aspect, there is provided a system for measuring electrophysiological readings comprising at least one electrode according the second or third aspect.

In some embodiments, the system is an EEG, EKG, EMG or EOG machine.

Other aspects, features, and advantages will become apparent from the following detailed description when taken in conjunction with the accompanying drawings, which are a part of this disclosure and which illustrate, by way of example, principles of the inventions disclosed.

### BRIEF DESCRIPTION OF THE FIGURES

The present disclosure will become better understood from the following detailed description of various non-limiting embodiments thereof, described in connection with the accompanying figures, wherein:

FIGS. 1A and 1B show diagrams of embodiments of graphene based electrodes for EEG according to the present invention.

FIG. 2 shows an SEM image of the graphene surface of an embodiment of a graphene based electrode according to the present invention.

FIG. 3 shows the EDS results for an embodiment of a graphene based electrode according to the present invention.

FIG. 4 shows the Raman spectra for an embodiment of a graphene based electrode according to the present invention.

FIG. 5 shows a Nyquist plot for repeated tests of an embodiment of a graphene based electrode according to the present invention.

FIG. 6 shows a Nyquist plot comparing the response of the graphene based electrode against other commercial sensors.

FIGS. 7A and 7B show Bode plots for repeated tests of an embodiment of a graphene based electrode according to the present invention.

FIG. 8 shows an SEM image of the graphene surface of a graphene based electrode for EEG according to the present invention following repeated testing.

FIG. 9 shows the EDS results for an embodiment of a graphene based electrode according to the present invention following repeated testing.

FIG. 10 shows the Raman spectra for an embodiment of a graphene based electrode according to the present invention prior to and following testing.

FIGS. 11A and 11B show XPS spectra for an embodiment of a graphene based electrode according to the present invention prior to and following testing. FIGS. 11C and 11D show the C1S deconvolution of said XPS results, and FIGS. 11E and 11F show the O1s deconvolution of said XPS results.

FIG. 12 shows the impedance for repeated tests of a graphene based electrode sensor on skin, including interruption of the skin/electrode contact.

FIG. 13 shows the impedance for repeated tests of a graphene based electrode sensor on a saline soaked wet cloth, including interruption of the saline soaked wet cloth/electrode contact.

FIG. 14A and 14B show the cyclic voltammograms and electrochemical impedance spectroscopy spectra respectively of bare silicon carbide film on highly doped silicon, epitaxial graphene, and epitaxial graphene following 100 tests.

FIG. 15 shows a diagram showing the proposed mechanism of surface conditioning at the graphene surface.

FIG. 16 shows a sample of EEG readings for commercial foam sensors.

FIG. 17 shows a sample of EEG readings for commercial spring based sensors.

FIG. 18 shows a sample of EEG readings for sensors formed from an embodiment of graphene based electrodes according to the present invention.

### DETAILED DESCRIPTION

In the following description, the electrodes will be described in reference to sensors electrodes for EEG applications for ease of understanding. It will be understood, however, that similar electrodes may be used for other applications where electrical activity is non-invasively measured through the skin, such as but not limited to ECG, EMG and EOG applications. Where appropriate, these measurements of electrical activity through the skin will be referred to as ‘electrophysiological readings.’

It will be understood that throughout this specification, this term relates to measurements taken at a relatively large scale and in a non-invasive manner by attachment of electrodes to the skin of a patient in a predetermined location chosen based off the organ or tissue of interest.

The inventors have previously created a method for producing graphene layers on silicon carbide which is described in US20160230304. In brief, the method involves depositing at least two metals onto a surface of SiC, and then heating the SiC and metal layers to cause at least one of the metals to react with the silicon in the SiC to form stable silicides as well as carbon, the metals being chosen so that their solubilities are low enough for the carbon to form a graphene layer between the silicide and the remaining SiC.

The inventors have used this technique to fabricate graphene based electrodes which are suitable for EEG and other electrophysiological reading applications. The inventors have found that in order to obtain suitable impedance, the graphene surface must undergo a conditioning step which can be carried out by repeated exposure to skin and air. The inventors have found that this conditioning step improves the impedance performance of the sensors, enabling their use in applications such as EEG, EKG, EMG and EOG.

The present disclosure will become better understood from the following example of a non-limiting embodiment.

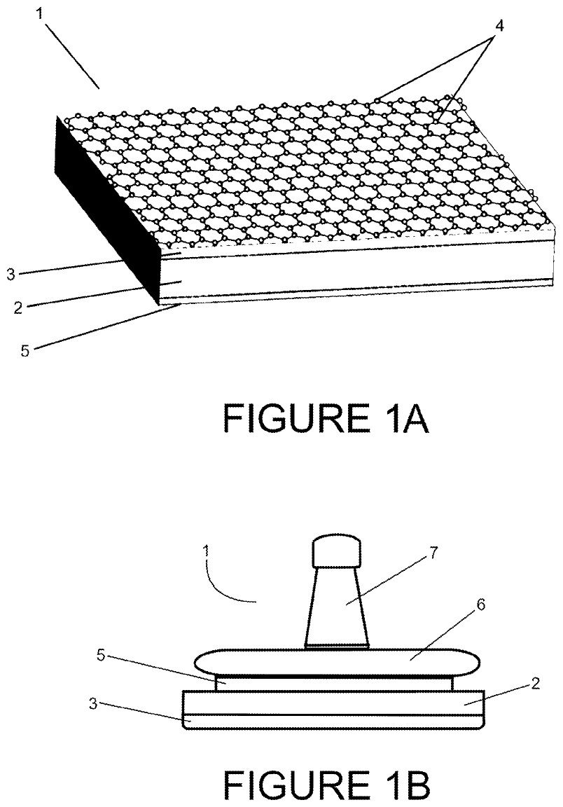

FIG. 1A shows a schematic diagram of an embodiment of a graphene based dry electrode for EEG applications. The electrode 1, also sometimes referred to as a biosensor, is composed of a highly doped silicon substrate 2, on which a film of silicon carbide 3 has been epitaxially grown. In this embodiment, the silicon carbide is in the form of 3C—SiC polytype. A surface 4 of this silicon carbide film has been graphitized, that is to say, the top surface 4 of the electrode includes at least one graphene layer. On the opposing side of the doped silicon substrate, an electrical contact 5 is provided so as to allow transmission of the electrophysiological readings. In preferred embodiments, the electrical contact is in the form of carbon tape, however it will be understood that other contacts, such as copper tape for example, may also be used. In this embodiment, the graphene surface has a rectangular shape, however in other embodiments, the surface may take another shape. This may be achieved by selectively etching the SiC layer prior to spluttering metallic layers, so as to only form graphene in the predetermined areas.

The silicon substrate has been highly doped in order to increase the conductivity of the substrate and maintain conductivity from the graphene surface 4 through the silicon carbide film 3, substrate 2 and electrical contact 5, where it can be communicated to an analyser through wiring or other standard methods. Known methods of doping silicon, such as with nitrogen or phosphorous may be used to increase the conductivity of the silicon substrate. In embodiments where the silicon substrate has not been sufficiently doped, a metallic layer or other electrical contact may extend around or through the substrate from the graphene surface to allow electrical communication with an analyser.

Sample electrodes were created using 3C—SiC films with a thickness of around 500 nm epitaxially grown on highly doped Si(100) substrate by placing them in a cryopump deposition chamber operating with CD Ar⁺ions and 200 mA current and spluttering a Ni layer of around 10 nm and a Cu layer of around 20 nm onto the film surface. The samples were then annealed by heating to around 1100° C. for one hour under vacuum conditions of around 10⁻⁵ mbar. This causes the breaking of Si—C bonds in the SiC and the release of carbon atoms which form a graphene layer on the surface, as well as nickel silicides in the metallic layer. These silicides, as well as any metal residues, were then removed by wet etching with Freckle solution for 9 hours. The conductivity of the surfaces was measured and found to be between ˜3 to 8 kΩ/square.

These electrodes were then mounted on foam covered by a copper tape. The foam was added in order to provide a mechanical pressure on the electrode towards the skin, ensuring effective contact with the skin, while the copper tape acts as an electrical contact for the electrode. The copper tape covering the foam backing may be the same copper tape that forms the electrical contact in the diagram of FIG. 1A. The produced electrodes had an effective area of 1 cm².

The inventors have further refined the electrode design as shown in FIG. 1B. In this embodiment, no foam backing is required and the substrate 2 is instead mounted on a metal pin button 6 using double sided carbon tape 5. In this way, electrical communication can occur from the skin of a patient and the graphene surface 3, through the highly doped silicon substrate 2 and carbon tape 5 and into the metal pin button 6, which due to the protruding pin 7 can then be connected to recording equipment by means of an alligator clip or other similar known method. In other embodiments, the metal pin may be soldered or otherwise electrically connected to wires or cables for connecting to recording equipment, or the metal pin button may be replaced with another form of electrically conducting surface connectable to recording equipment through wires or cables.

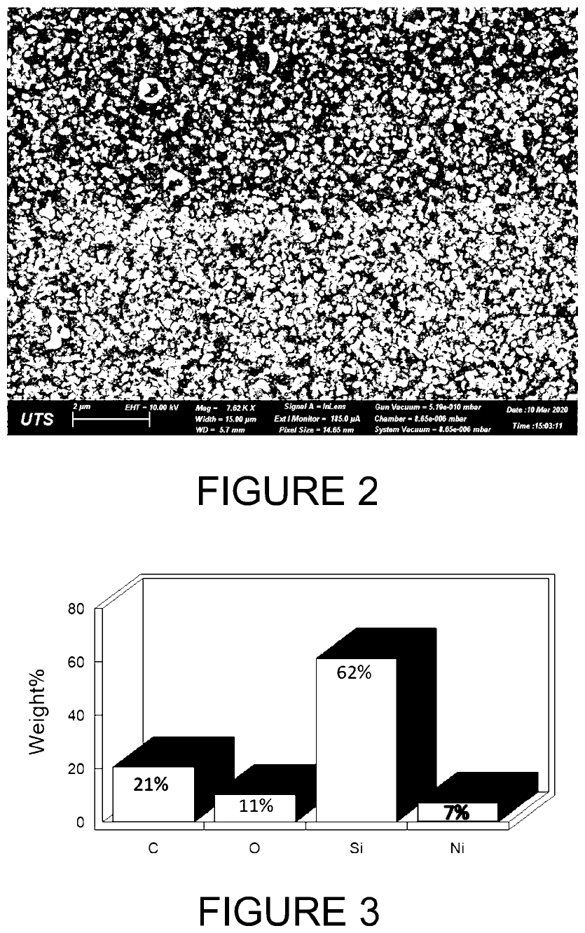

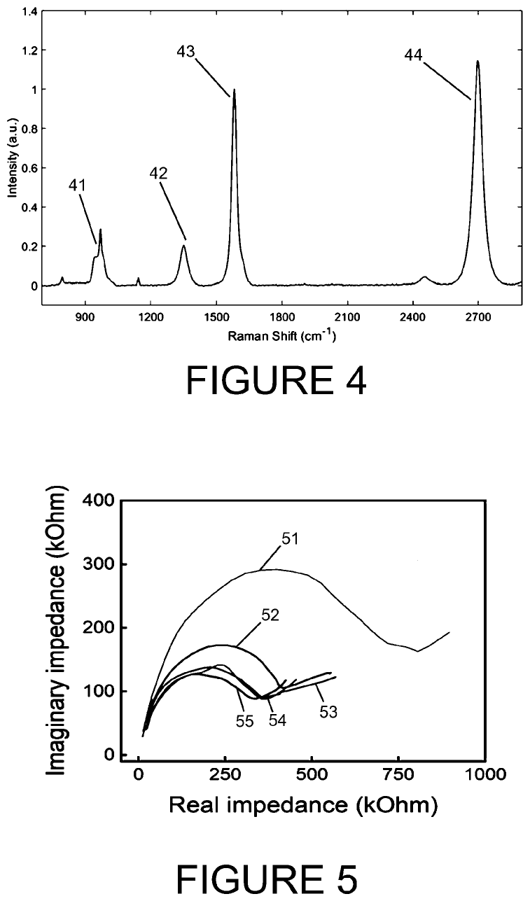

The presence of graphene on the electrodes was verified and characterized by scanning electron microscopy (SEM), energy-dispersive X-ray spectroscopy (EDS), Raman spectroscopy, and X-ray photoelectron spectroscopy (XPS). An SEM image, taken at a magnification of 7.62 kX is shown in FIG. 2. This SEM image shows a 15 μm by 15 μm area of the surface of the epitaxially grown graphene layer. The quantitative results of the EDS analysis, shown as weight % are shown in FIG. 3, and align with the expected values for a graphene surface on a silicon carbide film. The Raman spectra, shown in FIG. 4, shows four dominant peaks: the LO peak 41 of SiC at ˜970 cm⁻¹, and the graphene related D peak 42, G peak 43 and 2D peak 44 at ˜1340 cm⁻¹, ˜1580 cm⁻¹, and ˜2680 cm⁻¹ respectively. As the D peak arises from defects within the graphene lattice, the ratio of intensities of the D and G peaks can be used to assess the defect density of the material. The graphene was found to have an I_{D}/I_{G} ratio of 0.2, indicating the presence of high quality (low-defect) graphene with an average graphene grain size of ˜90 nm. The I_{2D}/I_{G} ratio may be used as an indicator of the graphene thickness. The I2D/IG ratio was determined to be 1.15, indicating the presence of 1-2 layers of graphene on the surface of the electrode. XPS data was collected using a Specs PHOIBOS 100 analyser operated with an Mg Kα X-ray source.

Experiments were then carried out to determine the behaviour of these graphene electrodes compared to dry electrodes made of a conductive polymer foam made of a urethane material, dry spring loaded pin electrodes as well as Ag/AgCl wet electrodes when used in electrophysiologic reading applications.

The first experiment measured the impedance of the sensors on human skin across a range of frequencies. This experiment used an impedance analyser with a three electrode configuration, with the graphene electrode being the working electrode, an Ag/AgCl wet electrode used as the reference electrode, and a gold electrode used as the counter electrode.

FIG. 5 shows a Nyquist plot of the frequency response for five successive tests 51, 52, 53, 54, 55 of the graphene based electrode. On the first test 51, one can see that there is a relatively large impedance. On the second test 52, however, the impedance is lower, and reduces further on the third test 53. On successive tests 54 and 55, the response stabilizes, and no further reduction in impedance is seen.

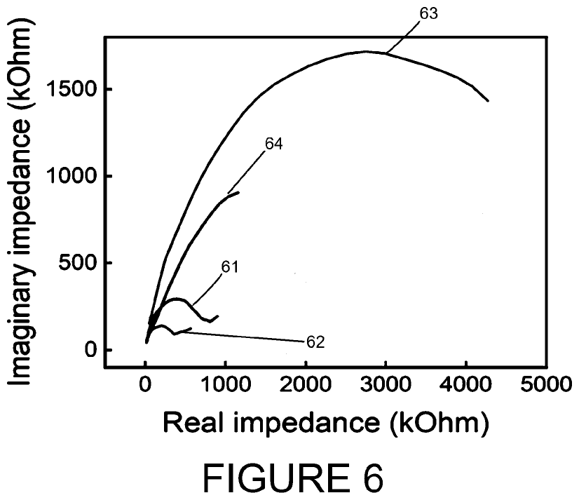

The performance of the electrodes compared to presently available EEG electrodes are shown in FIG. 6, which is a Nyquist plot comparing the first and third tests with other electrode materials. FIG. 6 shows the frequency response of the first test of the graphene electrode 61, the third test of the graphene electrode 62, a spring-loaded pin electrode 63, and a foam electrode 64. Both comparative electrodes were supplied by EEE Holter Technology Co, Ltd, Hsinchu, Taiwan. Electrodes according to the present invention show superior impedance performance relative to both the foam and spring-loaded pin electrode, though it should be noted that direct comparisons between the foam and graphene electrodes (which both have the same electrode area in contact with the skin), and the spring-loaded pin electrode which has pin-contacts. The change in impedance over the first three tests, as seen in the present graphene electrode, is not seen in the other electrodes.

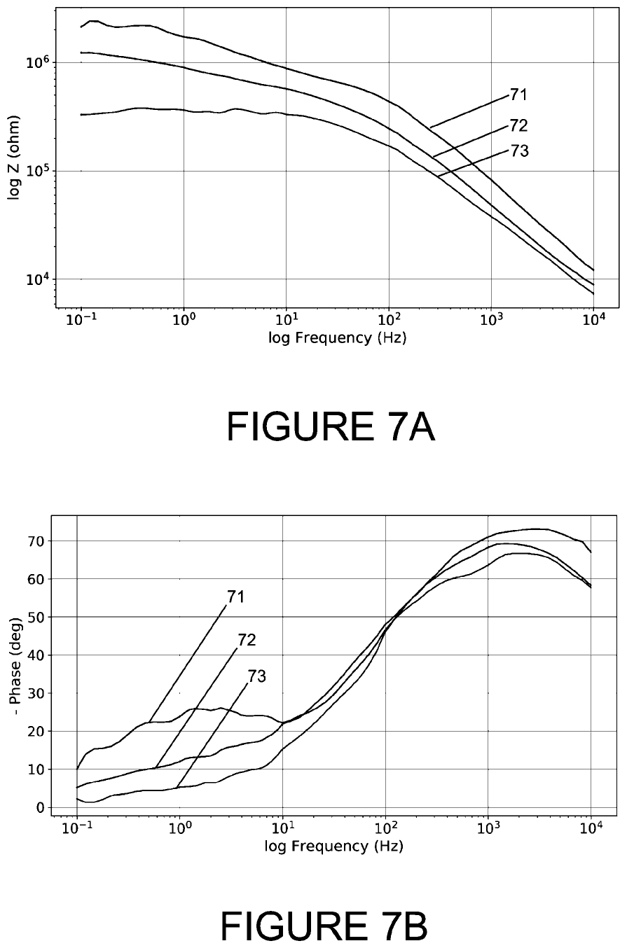

To further demonstrate the produced electrode's response to varying signals, and the effect of repeated testing of said electrodes, FIGS. 7A and 7B show Bode plots for the first test 71, second test 72, and third test 73 of the graphene electrode. The impedances at 10 and 50 Hz are tabulated below:

| | Z at 10 Hz (kΩ) | Z at 50 Hz (kΩ) |
|---|---|---|
| 1^{st} Test | 887 | 592 |
| 2^{nd} Test | 569 | 353 |
| 3^{rd} Test | 329 | 229 |

It is thus clear that there is a skin conditioning effect that occurs to the electrode as a result of repeated skin/air contact. To investigate the mechanism behind this change in impedance performance between the first and third tests, SEM imagery, EDS analysis, and Raman spectroscopy was carried out on an electrode following the surface conditioning effect in a similar fashion to those shown in FIGS. 2, 3 and 4 respectively.

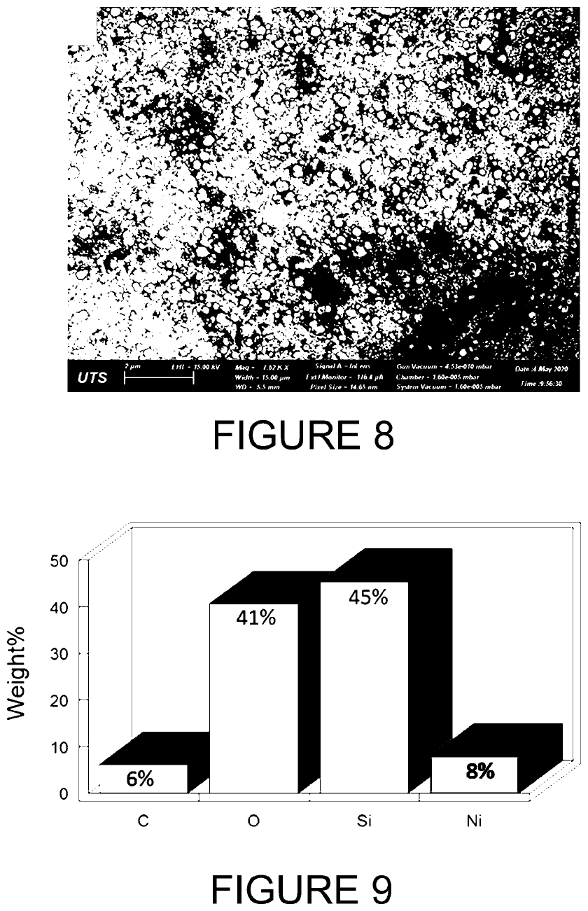

Comparing FIG. 2 which shows the pristine graphene surface (prior to testing) and FIG. 8 which shows the graphene surface following testing at the same microscope settings and magnification, it is clear that some degree of surface modification after repeated testing is notable.

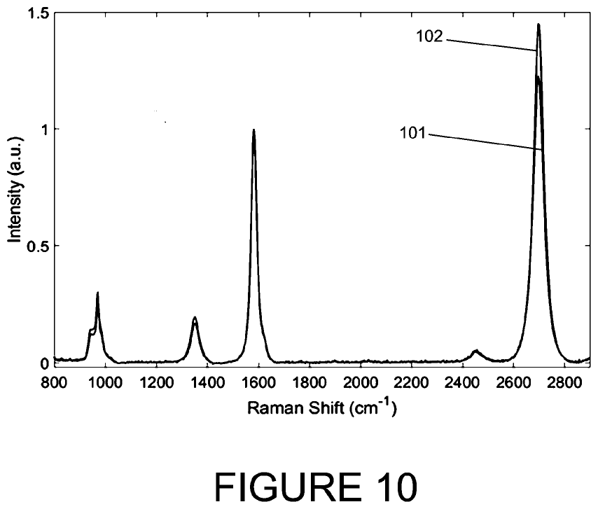

Comparing FIG. 3 and FIG. 9, which show the relative weight % of elements in the samples prior to and following testing respectively (as collected by EDS), a large increase in the amount of oxygen content can be seen following testing. FIG. 10 shows the Raman spectra of pristine graphene samples (101, in red) and following testing (102, in blue), which show that there has not been a large change in the Raman spectra over the course of testing.

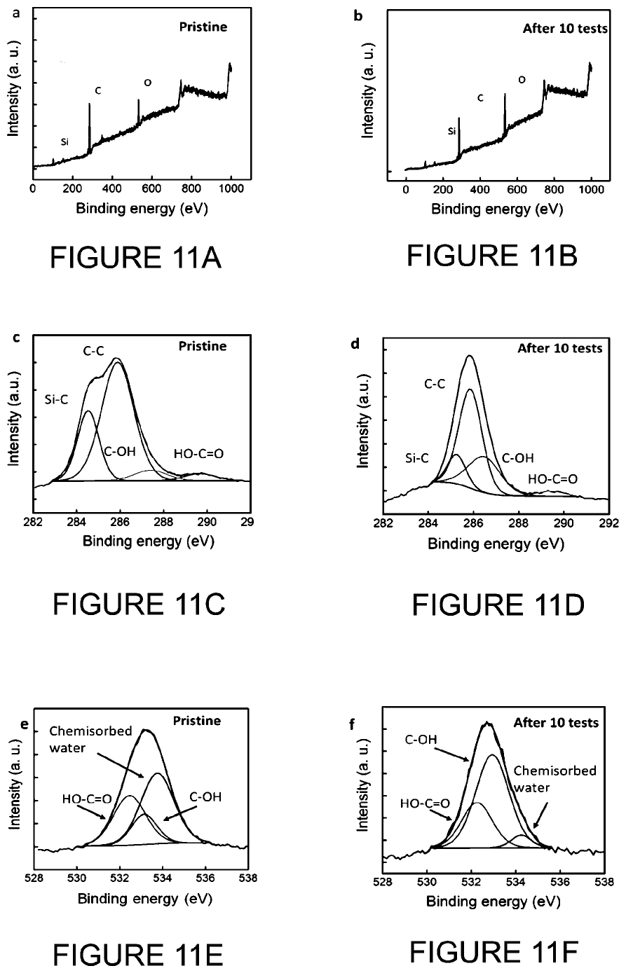

XPS analysis comparing samples of pristine (prior to testing) epitaxial graphene and samples of epitaxial graphene having undergone the skin conditioning step (following 10 repeated tests) are shown in FIGS. 11A to 11F. FIG. 11A shows the XPS survey analysis of a pristine epitaxial graphene surface and FIG. 11B shows the XPS survey analysis of the epitaxial graphene surface after 10 repeated tests. FIG. 11C and FIG. 11D show the CIS deconvolution of pristine graphene and graphene following 10 tests respectively. FIG. 11E and FIG. 11F show the O1s deconvolution of pristine graphene and graphene following 10 tests respectively. The XPS results showing the estimated at % for different elements from survey spectra, and identified bonds from the deconvolution of the C1s and O1s XPS peaks are tabulated below:

| Elements and | Pristine | After 10 sequential |
|---|---|---|
| bond types | (at. %) | tests (at. %) |
| C | 73 | 65.29 |
| Si | 11.88 | 13.23 |
| O | 15.12 | 21.48 |
| C1s Analysis | | |
| C═C | 65.1 | 55.95 |
| Si—C | 25.38 | 12.98 |
| C—OH | 5.85 | 27.89 |
| COOH | 3.71 | 3.17 |
| O1s Analysis | | |
| C—OH | 15.8 | 64.5 |
| COOH | 29.8 | 27.2 |
| Chemisorbed water | 54.4 | 8.3 |

Comparing these results, it appears that new oxygen species have emerged on the surface as corroborated by the EDS results, indicating that the graphene has undergone functionalisation during or as a result of the skin conditioning step. X

Looking at the C1s peak deconvolution for the pristine epitaxial graphene surface, there is an estimated 65.1, 25.4, 5.9 and 3.7 at % of C—C (graphenic), Si—C, C—OH (chemisorbed water) and COOH (carboxyl) bonds respectively. After ten repeated tests, a minor decrease of C—C bonds and a relatively high decrease in Si—C bonds was observed. The amount of COOH bonds remained roughly the same following testing, but the at % of C—OH bonds significantly increased. Without wishing to be bound by theory, it is surmised that these results indicate the formation of surface or edge functionalisation with hydroxyl groups on the epitaxial graphene following skin contact. The deconvolution of the O1s peak, as seen in FIGS. 11E and 11F, infers three types of oxygen bonds, COOH at 531.8 eV, C—OH at 533 eV, and chemisorbed water at 534.2 eV. The deconvolution of the O1s peak indicated at % ratios of approximately 29.8, 15.8 and 54.4 at % for COOH, C—OH bonds and chemisorbed water, respectively. Following ten repeated tests, the COOH amount remained roughly the same while C—OH bonds significantly increased and the amount of chemisorbed water decreased. While the formation of oxygen functional groups on the graphene surface typically leads to defects in the basal planes, this was not observed in the present surfaces as indicated by Raman spectroscopy (as shown by FIG. 10), where no significant change in the D peak intensity or I_{D}/I_{G} ratio occurred. This suggests that the formation of C—OH bonds observed by XPS results occurs mainly at the edges or grain boundaries of the graphene rather than the basal plane, which is attributed to the higher surface energy of the defective grain boundaries promoting the formation of functional groups.

The increase in C—OH and COOH bonds result in beneficial properties such as improved wettability of the electrodes, reduced contact impedance and a marked enhancement of the double-layer capacitance. Otherwise stated, the increased hydrophilicity (wetting) as a result of the functionalisation of the graphene is thought to lead to better electrolyte (sweat) penetration and ion intercalation, resulting in reduced contact impedance with the skin. Sweat is a body fluid containing a number of ions such as sodium, potassium, calcium, magnesium, chloride and lactate and functions as an electrolyte solution in this situation. Hydroxyl groups present in the sweat solution are thought to form C—OH bonds at the grain boundaries of the epitaxial graphene. Following this, water molecules in sweat are adsorbed on the surface (physisorption) starting from hydroxyls at the grain boundaries, and extend over time to cover partially or fully the grains. This forms a boundary layer which is held by weak van der Waals forces on the graphene surface. This theory is supported by XPS results which show a reduced intensity of C—C and Si—C bonds following repeated testing, indicating that a layer is formed on top of the graphene following skin contact.

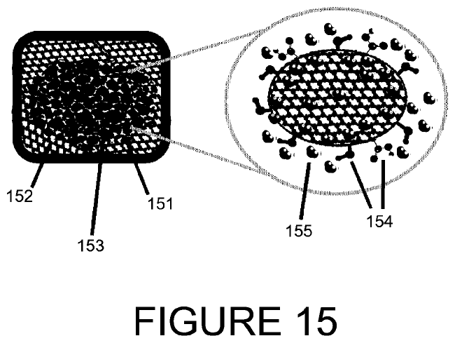

In summary, contact with the skin, or more specifically sweat on the skin, is thought to result in the formation of a semisolid boundary layer on top of the graphene surface and to lead to an improved wetting of the graphene surface and to allow ion intercalation in the graphene. Otherwise stated, it is thought that a thin layer of water molecules forms on the graphene surface through chemisorption and physisorption, facilitated by hydroxyl and carboxyl functional groups at the grain boundaries of epitaxial graphene. FIG. 15 shows a schematic diagram illustrating this proposed mechanism. A thin boundary layer 151 forms on the epitaxial graphene surface 152 (graphene grain boundaries 153 can be seen across the surface 152). The layer is formed as a result of hydroxyl and carboxyl functional groups 154 forming at the grain boundaries, allowing water molecules 155 to be adsorbed to cover the surface.

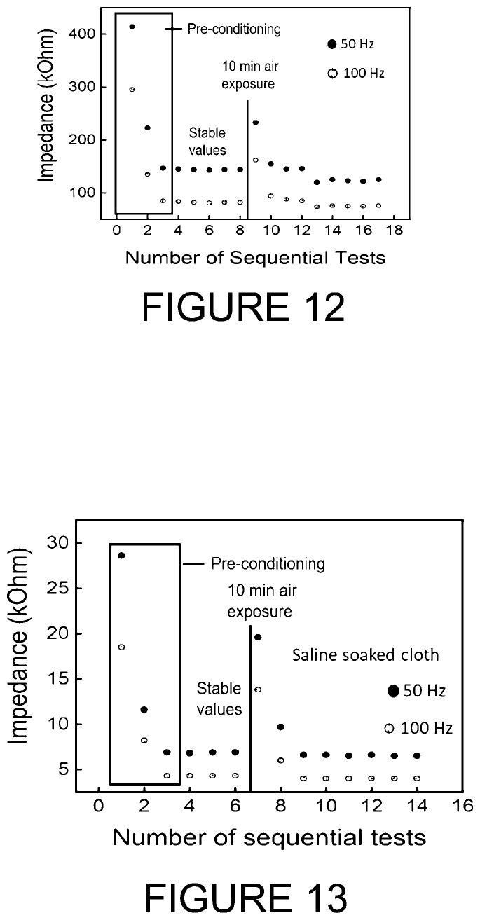

To further characterize the surface conditioning effect, the measured impedance at 50 and 100 Hz for ten repeated tests (without breaking skin contact) were plotted. These results are plotted in FIG. 12. These results show that the improvements associated with repeated testing (namely, a reduction in impedance) dropped off following the third successive test and substantially stable impedance values were measured for each successive test. Otherwise stated, these results show that three repeated tests is enough for the electrode to undergo the skin conditioning step and to establish a stable impedance value. To further understand the how the stability of the surface conditioning process changed over time, the electrodes were separated from skin contact and left exposed to the air for a period of 10 minutes, before resuming the tests. This shows that the contact impedance increases again after the contact with the skin is interrupted, likely due to the evaporation of the water boundary layer. However, it is possible to achieve low contact impedances following interruption by repeating the skin conditioning step/repeated testing using skin contact. Further, it appears that the surface conditioning occurs faster on successive skin conditioning steps following interruption. This indicates that it is likely that the additional C—OH functionalisation that takes place during the initial skin conditioning step remains following interruption, so the formation of the water boundary layer can occur immediately when placed in contact with a patient (and sweat) again.

To verify that the skin conditioning step is linked to the sweat of the patient, another similar experiment measuring the impedance at 50 and 100 Hz was conducted where the electrodes were placed on a wet cloth of saline water (0.01 M phosphate buffered saline solution) to model sweat rather than placed on human skin. Similar surface conditioning results were observed as for the skin contact. These results, in FIG. 13 show a similar skin conditioning process occurs as evidenced by a reduced contact impedance over three successive tests before a stable contact impedance is observed. As with the electrodes in skin contact, when removed from contact with the cloth and exposed to the air for ten minutes, the impedance rises on the next test, though this soon stabilizes to similar values as prior to the air exposure. This may provide an avenue for including a surface conditioning step during the manufacturing process. In preferred embodiments, an electrolyte solution, such as phosphate buffered saline solution) is used to perform the surface conditioning step.

To summarise the theorized mechanism of surface conditioning, it is thought that the graphene surface undergoes functionalisation, in particular the formation of C—OH and COOH bonds at the grain boundaries of the graphene due to repeated contact with the skin and in particular sweat which acts as an electrolyte. It is thought that the mechanism for reducing contact impedance between the skin and electrode involves the formation of a thin layer of electrolyte on the epitaxial graphene surface that forms a semisolid surface type of contact during sensing applications. This may cause the graphene structure to become more electrochemically active, resulting in enhanced skin and electrode interaction and a lower contact impedance which is required for successful EEG sensors. This effect may be simulated by conditioning the graphene surface using an electrolyte solution, preferably one which has similar properties to sweat, such as phosphate buffer solution.

An additional benefit of the aforementioned mechanism is that the graphene sensors do not appear to readily undergo delamination following exposure to sweat as would be expected. Rather, contrary to ordinary understanding, the sweat improves the performance of the present sensors by causing functionalisation at the grain boundaries.

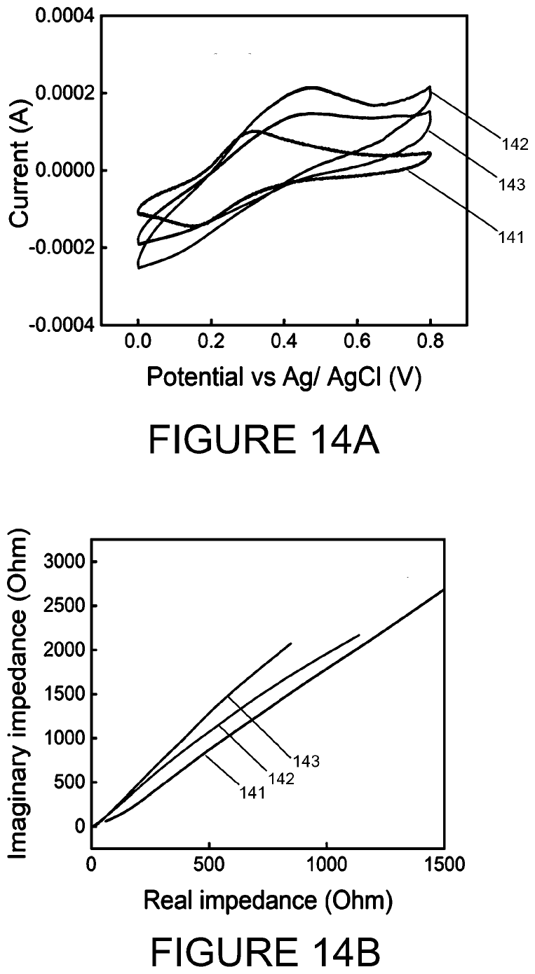

The electrochemical properties of epitaxial graphene and silicon carbide film on highly doped silicon in 0.1 M NaCl electrolyte (to simulate human sweat) were analysed by performing cyclic voltammograms (CV) at potential limits of 0.8 V to 0.0 V against Ag/AgCl electrodes in a three electrode system, at a scan rate of 100 mV/s. The results are shown in FIG. 14A, which shows the response of SiC film on highly doped silicon 141, epitaxial graphene 142, and the same epitaxial graphene after 100 repeated cycles 143. These curves show that epitaxial graphene possesses a substantially enhanced capacitive behaviour compared to the SiC film on highly doped silicon (about 1.8 times). Observing the CV curve of SiC shows redox peaks (oxidation peak at 0.28 V and reduction peak at 0.18 V), which is thought to be due to the presence of metal impurities. The CV curve of epitaxial graphene shows a slightly resistive behaviour, which is reduced upon repeated cycling (as evidenced by the response of the graphene following 100 repeated cycles 143).

To quantify and compare the transfer impedance between the electrodes and the electrolyte, electrochemical impedance spectroscopy (EIS) measurements were conducted in the 0.01 Hz to 100 kHz frequency range with a signal amplitude of 5 mV. The spectra of SiC film on highly doped silicon 141, epitaxial graphene 142, and the same epitaxial graphene after 100 repeated cycles 143. These curves show that the transfer impedance of the epitaxial graphene improves after 100 cycles, compared to both the initial epitaxial graphene and the SiC film reference. The measured charge transfer impedance (R_{ct}) for SiC on silicon was ˜160Ω, whereas the initial R_{ct} of the epitaxial graphene was ˜60Ω, reducing to ˜20Ω after 100 cycles.

The created graphene electrodes were also tested for their suitability for obtaining EEG by inserting them into a brain interface machine. Specifically, a headset with sensors at the locations Fp1, Fp2, Fz, C3, C4, Pz, O1 and O2 (according to the international 10-20 system) had the forehead sensors Fp 1 and Fp2 swapped with the created graphene electrodes. The impedance read by the headset was around 470 kΩ, compared with 421 kΩ and 300 kΩ for commercial foam based electrodes and commercial spring loaded electrodes respectively. The performance of the electrodes, as measured by impedance, could be improved by providing a gentle pressure or force on the electrode towards the skin, for example by securing the electrode to the skin by means of an elastic head band. Otherwise stated, it was observed that when electrodes were secured to the skin by an elastic head band, a lower contact impedance was measured.

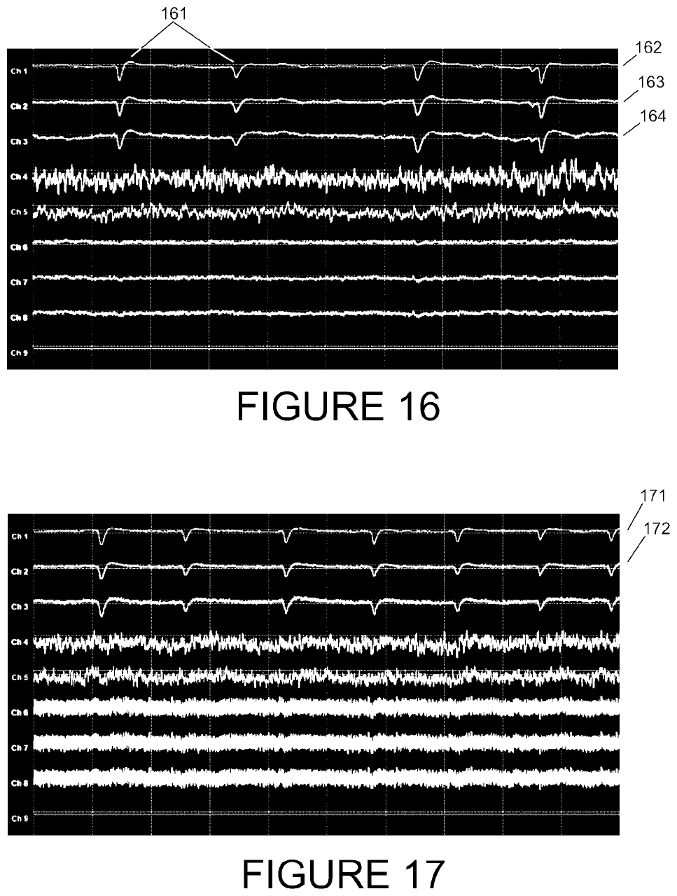

FIG. 16 shows a sample of EEG readings with commercial foam sensors in all locations. The subject was instructed to blink which produced a blink signal 161 which was picked up by the sensors in channels 1, 2 and 3 162, 163, 164.

FIG. 17 shows the same set up with the forehead sensors Fp 1 and Fp2, corresponding to channel 1 and channel 2 (171 and 172 respectively) replaced with commercial spring loaded pin sensors. The remaining sensors are all foam based as in FIG. 11. The blink signal produced by the subject blinking can be observed in both channel 1 and 2 (171 and 172 respectively).

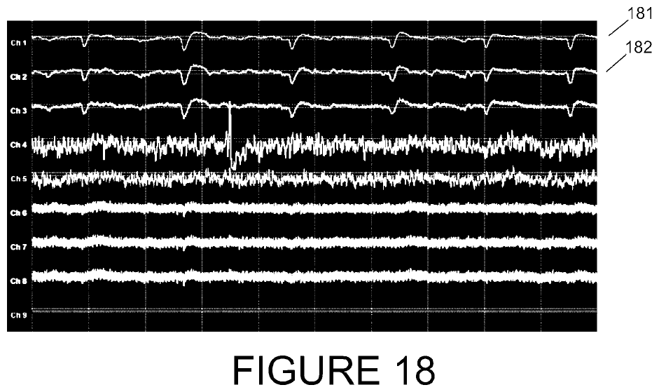

FIG. 18 shows EEG readings from the same set up as FIG. 17 except that dry graphene sensors according to the present invention have replaced the forehead sensors Fp1 and Fp2, corresponding to channel 1 and channel 2 (181 and 182 respectively). The blink signal was successfully picked up by the graphene electrodes in a similar manner to the two commercially available electrodes as shown in FIG. 16 and FIG. 17. This shows that the produced electrodes are suitable for use in gathering EEG readings. Additionally, a problem with existing polymer-graphene based electrodes is that their performance degrades over time, often because of the graphene's delamination. In the produced electrodes, however, the exposure to sweat causes a skin conditioning step without any delamination being observed.

In the foregoing description of certain embodiments, specific terminology has been resorted to for the sake of clarity. However, the disclosure is not intended to be limited to the specific terms so selected, and it is to be understood that each specific term includes other technical equivalents which operate in a similar manner to accomplish a similar technical purpose.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The reference in this specification to any prior publication (or information derived from it), or to any matter which is known, is not, and should not be taken as, an acknowledgement or admission or any form of suggestion that prior publication (or information derived from it) or known matter forms part of the common general knowledge in the field of endeavour to which this specification relates.

In addition, the foregoing describes only some embodiments of the invention(s), and alterations, modifications, additions and/or changes can be made thereto without departing from the scope and spirit of the disclosed embodiments, the embodiments being illustrative and not restrictive.

Furthermore, invention(s) have described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention(s). Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment.

1. A process for forming a dry electrode for measuring electrophysiological readings, comprising:
epitaxially growing a silicon carbide film on a doped silicon substrate;
depositing at least two metals on a surface of the silicon carbide film, the at least two metals including at least one first metal and at least one second metal;
heating the at least two metals, silicon carbide film, and substrate to cause the at least one first metal to react with silicon of the silicon carbide film to form carbon and at least one stable silicide, and the corresponding solubilities of the carbon in the at least one stable silicide and in the at least one second metal are sufficiently low that the carbon produced by the silicide reaction forms a graphene layer on the silicon carbide film;
removing the at least one stable silicide and unreacted at least first and second metals to produce a structure having a doped silicon substrate and a silicon carbide film with a surface layer of graphene;
repeatedly contacting the surface layer of graphene with an electrolyte solution to condition the graphene surface prior to use.

2. The process of claim 1, wherein the composition of the electrolyte solution is similar to human sweat.

3. The process of claim 1, wherein the electrolyte solution is phosphate buffered saline solution.

4. The process of claim 3, wherein the concentration of the phosphate buffered saline solution is 0.01M.

5. The process of claim 1, wherein the electrolyte is human sweat.

6. The process of any one of claim 1, wherein the corresponding solubility of carbon in the at least one second metal is lower than the corresponding solubility of carbon in the at least one stable silicide.

7. The process of any one of claim 1, wherein the at least one first metal is nickel, and the at least one second metal is copper.

8. (canceled)

9. The process of any one of claim 1, wherein the heating step is performed under vacuum wherein the vacuum has a pressure of about 10⁻³ to 10⁻⁵ mbar.

10. (canceled)

11. The process claim 1, wherein the heating step is carried out above 800° C.

12. The process of claim 11, wherein the heating step is carried out at about 1100° C.

13. (canceled)

14. The process of claim 1 wherein the step of contacting the surface layer of graphene with the electrolyte solution is repeated between three and ten times.

15. A dry electrode for measuring electrophysiological readings, comprising:
a doped silicon substrate;
a silicon carbide film on the substrate;
a graphene surface on the silicon carbide film;
wherein the graphene surface has undergone a conditioning step of repeatedly contacting the surface layer of graphene with an electrolyte solution prior to use.

16. A dry electrode for measuring electrophysiological readings, comprising:
a doped silicon substrate;
a silicon carbide film on the substrate;
a graphene surface on the silicon carbide film;
wherein the graphene surface has undergone a functionalisation and/or intercalation process to increase the amount of oxygen functional groups present.

17. The dry electrode of claim 16, wherein the oxygen functional groups include C—OH and COOH.

18. The dry electrode of claim 16, wherein the functionalisation and/or intercalation process occurs substantially at the grain boundaries of the graphene surface.

19. The dry electrode claim 16, wherein the substrate is placed in electrical communication with a metal pin button.

20. The dry electrode of claim 19, wherein the substrate is attached to the metal pin button by carbon tape.

21. (canceled)

22. A system for measuring electrophysiological readings comprising at least one electrode according to claim 16.

23. The system of claim 22, wherein the system is an EEG, EKG, EMG or EOG machine.
